(19) United States
(12) Patent Application Publication
(10) Pub. No.: US 2026/0108868 A1
(43) Pub. Date: Apr. 23, 2026

(54) MFI ZEOLITES WITH SUBSTITUTED HETEROATOMS FOR SELECTIVE TOLUENE METHYLATION

(71) Applicant: PURDUE RESEARCH FOUNDATION, West Lafayette, IN (US)

(72) Inventors: Rajamani P. Gounder, West Lafayette, IN (US); Andrew T. Norfleet, Kawkawlin, MI (US); Bereket Tassew Bekele, West Lafayette, IN (US)

(21) Appl. No.: 19/362,649

(22) Filed: Oct. 20, 2025

Related U.S. Application Data
(60) Provisional application No. 63/708,768, filed on Oct. 18, 2024.

(51) Int. Cl. B01J 29/40; B01J 37/30; C01B 39/40; C07C 2/86

(52) U.S. Cl. CPC B01J 29/40; B01J 37/30; C01B 39/40; C07C 2/865; C07C 2529/40; C07C 2529/46

(57) ABSTRACT

A process for selective aromatic methylation using improved MFI zeolites and methods for making the same. The process contacts one or more aromatics and one or more oxygenates with a MFI zeolite comprising less than 30 wt % aluminum at reaction conditions sufficient to produce a product comprising one or more xylenes. The MFI zeolite can be obtained by combining a unique combination of one or more structure directing agents (SDA) and one or more heteroatoms, T, which produce a product slate that is highly selective toward para-xylenes.

### GOVERNMENT LICENSE RIGHTS

This invention was made with government support under Cooperative Agreement No. EEC-1647722 awarded by the National Science Foundation. The government has certain rights in the invention.

### BACKGROUND

### Field of the Invention

Embodiments of the present invention generally relate to the synthesis of zeolite catalysts and methods for using the same for selectively converting toluene to para-xylene (“p-X”).

### Description of the Related Art

Zeolites are inorganic silica-based solids with microporous void structures that impart shape selectivity on reactants, transition states, and products, leading them to catalyze chemical reactions with unique reactivities and selectivities. These structures contain tetrahedral silicon atoms linked by oxygen, forming charge-neutral crystalline networks. The isomorphous substitution of framework Si-atoms with trivalent atoms (“T”), such as Al³⁺, Ga³⁺, Fe³⁺, or B³⁺ creates anionic charges that can be compensated by protons located on bridging 0-atoms (e.g., Al—O(H)—Si), which act as Brønsted acid sites for catalysis.

MFI zeolites are a crystalline aluminosilicate material with a three-dimensional framework of straight and zigzag channels that are crossed with each other. MFI zeolites are acidic catalysts and widely used in industrial catalytic applications for obtaining para-xylene (p-X), a valuable polymer precursor, through toluene methylation by dimethyl ether (DME) or methanol, while the ortho-xylene (o-X) and meta-xylene (m-X) isomers are undesired side-products. Production of p-X on acid catalysts through toluene methylation is limited because p-X is a minor product in both thermodynamic equilibrium (˜50% m-X, ˜25% p-X, ˜25% o-X; 573-673 K) and kinetic (˜60% o-X, ˜30% p-X, ˜10% m-X) controlled reaction conditions.

The synthetic placement of aluminum (Al) atoms and, consequently, H⁺ sites among channel and channel intersection environments of MFI zeolites varies with the structure-directing agents (SDAs) used during synthesis. Active site design approaches in MFI that systematically bias sites towards smaller (0.55 nm) straight and sinusoidal channels (compared to channel intersections—0.7 nm) have been proposed to increase p-X selectivity (˜80%) heralding an approach for intrinsic kinetically controlled production of p-X.

One example of a conventional organic SDA (OSDA) that has been used to crystallize MFI is tetra-n-propylammonium (TPA⁺), which contains a quaternary N⁺ center that is occluded in channel intersections and confers energetic preference to position Al in T-sites closest to this N⁺ center, thus resulting in crystals with H⁺ sites biased towards channel intersections.

Non-conventional SDAs, such as ethylenediamine (EDA) along with TPA or a mixture of 1,4-diazabicyclo[2.2.2]octane (DABCO) and methylamine (MA) as co-SDAs, have also been used to crystallize MFI. These non-conventional SDAs provide hydrogen bonding interactions that bias Al in T-sites away from the N⁺ atom and towards the straight and sinusoidal channels. Such non-conventional SDAs used for aluminosilicate zeolites have showed an increase in p-X selectivity.

### REFERENCES OF INTEREST INCLUDE

Ezenwa, S.; Locht, H.; Montalvo-Castro, H.; Hoffman, A.; Attebery, J.; Jan, D.-Y.; Schmidthorst, M.; Chmelka, B.; Hibbitts, D.; Gounder, R. Synthetic Placement of Active Sites in Zeolites for Selective Toluene Methylation to Para-Xylene. Journal of the American Chemical Society 2024, 146, 10666-10678, https://dx.doi.org/10.1021/jacs.4c00373.
Jan, D.-Y.; Gounder, R.; Ezenwa, S.; Moscoso, J. G.; Kuzmanich, G. Mfi Zeolite of Highly Dispersed Framework Aluminum and Its Uses for Selective Aromatics Methylation to Para-Xylene. US20230278017A1, Sep. 7, 2023. https://patents.google.com/patent/US20230278017A1/en (accessed 2024 Jun. 4).
Miller, J. T.; Gounder, R.; Ribeiro, F. H.; Tseng, H.-T.; Kester, P. M.; Hur, Y. G.; Cho, Y. R. Increased Oligomer Selectivity from Olefin Oligomerization by Incorporation of Boron. U.S. Pat. No. 11,358,912B2, Jun. 14, 2022. https://patents.google.com/patent/US11358912B2/en (accessed 2024 Aug. 27).
Jones, A. J.; Carr, R. T.; Zones, S. I.; Iglesia, E. Acid Strength and Solvation in Catalysis by MFI Zeolites and Effects of the Identity, Concentration and Location of Framework Heteroatoms. Journal of Catalysis 2014, 312, 58-68. https://doi.org/10.1016/j.jcat.2014.01.007.
Hur, Y. G.; Kester, P. M.; Nimlos, C. T.; Cho, Y.; Miller, J. T.; Gounder, R. Influence of Tetrapropylammonium and Ethylenediamine Structure-Directing Agents on the Framework Al Distribution in B—Al-MFI Zeolites. Ind. Eng. Chem. Res. 2019, 58 (27), 11849-11860. https://doi.org/10.1021/acs.iecr.9b01726.
Kester, P. M.; Miller, J. T.; Gounder, R. Ammonia Titration Methods To Quantify Brønsted Acid Sites in Zeolites Substituted with Aluminum and Boron Heteroatoms. Ind. Eng. Chem. Res. 2018, 57 (19), 6673-6683. https://doi.org/10.1021/acs.iecr.8b00933.
Hill, I.; Malek, A.; Bhan, A. Kinetics and Mechanism of Benzene, Toluene, and Xylene Methylation over H-MFI. ACS Catal. 2013, 3 (9), 1992-2001. https://doi.org/10.1021/cs400377b.

There is still a need for new methods for synthesizing MFI zeolites and methods for aromatic methylation that provide improvement in p-X selectivity.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are, therefore, not to be considered limiting of its scope, for the invention may admit to other equally effective embodiments. It is emphasized that the figures are not necessarily to scale and certain features and certain views of the figures can be shown exaggerated in scale or in schematic for clarity and/or conciseness.

FIGS. 1A-1F depict individual xylenes formation rates as a function of aromatic pressures on Fe-MFI-TPA (FIG. 1A), (B)—Fe-MFI-EDA/TPA (FIG. 1B), Ga-MFI-TPA (FIG. 1C), (B)—Ga-MFI-EDA/TPA (FIG. 1D), B-MFI-TPA (FIG. 1E), and B-MFI-EDA/TPA (FIG. 1F). Reaction conditions: 130° C., DME partial pressure 66 kPa, space velocities of 0.0016-0.22 mol_{Toluene} mol_{H+}⁻¹s⁻¹.

FIG. 2 depicts zero-order xylenes selectivity across synthesis methods (EDA/TPA) and heteroatoms (Al, Fe, Ga, B). Reaction conditions: 130° C., DME partial pressure 66 kPa, space velocities of 0.0016-0.22 mol_{Toluene} mol_{H+}⁻¹s⁻¹.

FIG. 3A depicts zero-order rate constants for total xylenes formation versus the DPE of various framework heteroatoms. Reaction conditions: 130° C., DME partial pressure 66 kPa, space velocities of 0.0016-0.22 mol_{Toluene} mol_{H+}⁻¹s⁻¹.

FIG. 3B depicts first-order rate constants for total xylenes formation versus the deprotonation energy (DPE) of various framework heteroatoms. Reaction conditions: 130° C., DME partial pressure 66 kPa, space velocities of 0.0016-0.22 mol_{Toluene} mol_{H+}⁻¹s⁻¹.

FIGS. 4A and 4B depict X-ray diffraction patterns for T-MFI-TPA and (B)-T-MFI-EDA/TPA samples, respectively, synthesized with various framework heteroatoms.

FIGS. 5A and 5B depict X-ray diffraction patterns for Fe-MFI-EDA/TPA and Ga-MFI-EDA/TPA samples, respectively, synthesized without the addition of H₃BO₃ precusor.

FIGS. 6A and 6B depict N₂ physisorption measurements at 77 K measured on T-MFI-TPA and (B)-T-MFI-EDA/TPA samples, respectively, synthesized with various framework heteroatoms.

### SUMMARY OF THE INVENTION

A process for selective aromatic methylation using improved MFI zeolites and methods for making the same as provided. The process contacts one or more aromatics and one or more oxygenates with a MFI zeolite comprising less than 30 wt % aluminum at reaction conditions sufficient to produce a product comprising one or more xylenes. The MFI zeolite can be obtained by combining a unique combination of one or more structure directing agents (SDA) and one or more heteroatoms, T, and can be used to produce a product slate that is highly selective toward para-xylene. Preferred heteroatoms, T, include boron, gallium, iron, and combinations thereof.

In at least one specific embodiment, the process contacts one or more aromatics and one or more oxygenates with a MFI zeolite comprising less than 30 wt % aluminum at reaction conditions sufficient to produce a product comprising one or more xylenes. The MFI zeolite can be obtained by combining one or more structure directing agents (SDA) and water to form an aqueous solution; adding one or more heteroatoms, T, selected from the group consisting of boron, gallium, iron, and combinations thereof to the aqueous solution to form an intermediate agent; adding a source of silicon to the intermediate agent to form a synthesis gel; and crystallizing the synthesis gel to form the MFI zeolite.

### DETAILED DESCRIPTION

It is to be understood that the following disclosure describes several exemplary embodiments for implementing different features, structures, or functions of the invention. Exemplary embodiments of components, arrangements, and configurations are described below to simplify the present disclosure; however, these exemplary embodiments are provided merely as examples and are not intended to limit the scope of the invention. Additionally, the present disclosure can repeat reference numerals and/or letters in the various embodiments and across the figures provided herein. This repetition is for the purpose of simplicity and clarity and does not in itself dictate a relationship between the various embodiments and/or configurations. Moreover, the exemplary embodiments presented below can be combined in any combination of ways, i.e., any element from one exemplary embodiment can be used in any other exemplary embodiment, without departing from the scope of the disclosure.

Additionally, certain terms are used throughout the following description and claims to refer to particular components. As one skilled in the art will appreciate, various entities can refer to the same component by different names, and as such, the naming convention for the elements described herein is not intended to limit the scope of the invention, unless otherwise specifically defined herein. Further, the naming convention used herein is not intended to distinguish between components that differ in name but not function.

Furthermore, in the following discussion and in the claims, the terms “including” and “comprising” are used in an open-ended fashion, and thus should be interpreted to mean “including, but not limited to.” The phrases “consist essentially of” and “consisting essentially of” mean that the described/claimed composition does not include any other components that will materially alter its properties by any more than 5% of that property, and in any case does not include any other component to a level greater than 3 mass %.

Unless otherwise indicated, all numerical values are “about” or “approximately” the indicated value, meaning the values take into account experimental error, machine tolerances and other variations that would be expected by a person having ordinary skill in the art. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for making the measurement.

The term “or” is intended to encompass both exclusive and inclusive cases, i.e., “A or B” is intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins are used, unless specified to the contrary or the context clearly indicates that only one olefin is used.

The term “wt %” means percentage by weight, “vol %” means percentage by volume, “mol %” means percentage by mole, “ppm” means parts per million, and “ppm wt” and “ppmw” are used interchangeably and mean parts per million on a weight basis. All concentrations herein, unless otherwise stated, are expressed on the basis of the total amount of the composition in question.

Each of the appended claims defines a separate invention, which for infringement purposes is recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this disclosure is combined with publicly available information and technology.

Aluminum free (“Al-free”) MFI zeolite catalysts and methods for synthesizing the same are provided. It has been surprisingly and unexpectedly discovered that an Al-free zeolite can be made that can control the per-site rates that are selective for para-xylene (“p-X”) production. Instead of traditional aluminosilicates, the Al-free material can be made from one or more heteroatoms, including Ga, Fe, and B. The resulting Al-free MFI zeolites are highly selective for p-X production. Not wishing to be bound by theory, it is believed that because heteroatoms differ in acid strength, quantified by deprotonation energy (DPE), that the selection of active species permits control over the per-site rates that are intrinsic to the support material.

The terms “aluminum-free” and “Al-free” are used interchangeably herein and refer to zeolites that contain less than 30 wt % aluminum. In certain embodiments, the Al-free zeolites contain less than 25 wt %, less than 20 wt %, less than 15 wt %, less than 10 wt %, less than 5 wt %, less than 2.5 wt %, less than 1.0 wt %, less than 0.5 wt %, less than 0.01 wt %, or 0.0 wt %.

The Al-free zeolites can be synthesized with one more heteroatoms, T, selected from the group consisting of Ga, Fe, B and combinations thereof. The MFI zeolites can have high Si/T ratios and small crystallite sizes. For instance, the MFI zeolites can have Si/T ratios that range from 10,000:1 to 3:1. The MFI zeolites also can have Si/T ratios that range from 5,000:1 to 10:1; 1,000:1 to 100:1; or 50:1 to 600:1. The Si:T ratio also can range from a low of about 25, 30, 40, or 50 to high of about 100, 200, 300, or 600. The Si:T ratio also can range from a low of about 50, 85, 125, or 165 to a high of about 275, 350, 400, or 600. The Si:T ratio also can be of from 50-600, 50-500, or 40-400.

A preferred crystallite size is about 0.001 μm to about 1,000 μm, more preferably about 0.01 μm to about 0.05 μm. Other crystallite sizes (in microns) can range from a low of about 0.001, about 0.005, or about 0.01 to a high of about 500, about 750, or about 1,000.

Any suitable conventional SDA and/or non-conventional SDA can be used to synthesize the Al-free zeolites. For example, suitable SDAs can be or can include tetrapropylammonium (TPA), ethylenediamine (EDA), 1,4-Diazabicyclo[2.2.2]octane (DABCO), methylamine (MA), tert-butylamine, n-butylamine, tripropylamine, dipropylamine, tetraethylammonium, 1,2,4-Trimetyl-1H-pyrazole-2-ium (1,2,4-TMP), pentaertythritol (PET), and combinations or mixtures thereof.

According to one or more embodiments provided herein, Al-free ZSM-5 zeolites can be synthesized from Ga, Fe, and/or B heteroatoms with both conventional SDAs and non-conventional SDAs. It was surprisingly discovered that higher para-xylene selectivity was obtained during toluene methylation on (B)-T-MFI made with non-conventional OSDA compared to T-MFI (Zeolyst) and (B)-T-MFI samples made with conventional OSDA (where T indicates the isomorphous substitution of Si⁴⁺ by Ga³⁺, Fe³⁺, or B³⁺). On a per-site basis, it was discovered that total xylene formation rates inversely correlated with the deprotonation energy of substituent heteroatoms.

In at least one specific embodiment, the Al-free MFI zeolites provided herein can be synthesized with Ga, Fe, and/or B heteroatoms substitution using TPA as the sole SDA or a combination of TPA and EDA as co-SDAs. MFI zeolites synthesized with the combination of TPA and EDA demonstrated higher p-X selectivities (>80%) during toluene methylation compared to MFI zeolites synthesized using TPA only (p-X selectivity ˜30%). It has been surprisingly discovered that the combination of TPA and EDA as co-SDAs controls the positioning of the trivalent heteroatoms in the zeolite material, and synthesizes an MFI zeolite with active H⁺ sites that is suitable for use as an aromatic methylation catalyst that is intrinsically (kinetically) selective for p-X.

The total amount of SDA(s) per gram of silica can range from a low of about 0.5 gr, 0.6 gr, or 0.7 gr to a high of about 1.0 gr, 1.2 gr, or 1.4 gr. The total amount of SDA(s) per gram of silica can range from a low of about a low of about 0.3 gr, 0.4 gr, or 0.5 gr to a high of about 0.8 gr, 0.9 gr, or 1.0 gr.

The source of silicon can be colloidal silica, a silicon alkoxide compound, fumed silica, amorphous silica, aluminosilicate, and any combinations or derivatives thereof.

The MFI zeolites can be synthesized by adding one or more SDAs to water to form an aqueous solution; homogenizing the aqueous solution for a first time period; adding a source of the one or more heteroatoms to the homogenized aqueous solution to form an intermediate agent and homogenizing the intermediate agent for a second time period to form a heteroatom-containing intermediate agent; adding a source of silicon to the heteroatom-containing intermediate agent to form a heteroatom-silicate intermediate agent and homogenizing the heteroatom-silicate intermediate agent for a third time period to form a synthesis gel; subjecting the synthesis gel to a crystallization process to crystallize the MFI zeolite; and then recovering the solids (e.g., by centrifugation) followed by washing, drying and a high temperature air treatment and subsequent ion-exchange treatments to remove any unreacted reagents or SDA compounds, thereby recovering the acid-form zeolite.

Each of first, second and third time periods can be the same or different, and each can range from about 1 second to about 48 hours; or about 1 to about 20 hrs; or about 2 to about 10 hrs; or about 3 to about 8 hrs; or about 3 to about 5 hrs; or about 3 to about 4 hrs.

The crystallization process preferably occurs at about 130° C. to about 150° C., but can range from as low of about 60° C., 70° C., 80° C. or 90° C. to a high of about 150° C., 180° C., 200° C. or 240° C. The crystallization process can also take place at about 100° C., 130° C., 140° C., 150° C., 160° C., 170° C., or 180° C.

The high temperature air treatment can occur at about 450 to about 550° C. The air treatment but can also range from as low of about 400° C., 410° C., or 420° C. to a high of about 500° C., 550° C. or 600° C.

Embodiments provided herein leverage the selection of synthesis precursors, combined with trivalent heteroatoms of varying identity, to permit finer control of individual and total formation rates while maintaining the desired selectivity toward p-X using MFI zeolites. The resulting MFI zeolites possess acid site locations and acid strengths that allow for increasing or decreasing reaction rates (e.g., by varying the heteroatom identity) while maintaining high selectivites top-X.

The terms “xylene” or “xylenes”, as used herein, refer to the class of dimethyl benzene molecules that include 1,2-dimethylbenzene, 1,3-dimethylbenzene, and 1,4-dimethylbenzene. 1,2-dimethylbenzene is often referred to as ortho-xylene or o-X. 1,3-dimethylbenzene is often referred to as meta-xylene or m-X. 1,4-dimethylbenzene is often referred to as para-xylene or p-X.

The MFI zeolites provided herein are intrinsically more selective to para-xylene formation and less selective toward meta-xylene, ortho-xylene, and light olefins and paraffins formation. In one or more embodiments, a process for producing para-xylene using the MFI zeolite provided herein includes reacting one or more oxygenates with an aromatic feedstock that comprises toluene and/or benzene in a methylation zone operating under alkylation conditions that include a maximum temperature of about 400° C. to about 675° C. and a pressure of about 10 kPa to 5000 kPa in the presence of the MFI zeolites provided herein to provide a product stream comprising para-xylene.

The aromatic feedstock or feed stream can be or can include benzene and/or one or more alkylaromatic hydrocarbons of the general formula C₆H(₆₋ₙ)Rₙ, where n is an integer from 0 to 5 and each R may be CH₃, C₂H₅, C₃H₇, or C₄H₉, in any combination. The aromatic feed stream can be derived from a variety of sources, including without limitation conventional catalytic reforming of C₆-C₇ non-aromatics from light naphtha or aromatic extraction raffinates to benzene and toluene, steam pyrolysis of naphtha, distillates or other hydrocarbons to yield light olefins and aromatics-rich byproducts (including gasoline-range material often referred to as “pygas”), and catalytic or thermal cracking of distillates and heavy oils to yield products in the gasoline range. Products from pyrolysis or other cracking operations generally will be hydrotreated according to known processes before being charged to the complex in order to remove sulfur, olefins and other compounds which would affect product quality and/or damage catalysts and downstream process. Light cycle oil from catalytic cracking also can be beneficially hydrotreated and/or hydrocracked according to known processing techniques to yield products in the gasoline range; the hydrotreating preferably also applies to catalytic reforming to yield the aromatic feed stream.

In one or more embodiments, the aromatic feed stream may be or may include benzene. In one or more embodiments, the aromatic feed stream may be or may include toluene. In one or more embodiments, the aromatic feed stream may be or may include both benzene and toluene.

The process condition for formation of para-xylene may include a maximum temperature of from about of about 400° C. to about 675° C., preferably from about 450° C. to about 650° C. and more preferably from about 500° C. to about 625° C. In accordance with various embodiments, the maximum temperature may refer to the maximum temperature of the catalyst bed and may be interchangeably referred to as the maximum bed temperature.

The process condition for formation of para-xylene may also include a pressure of from about 10 kPa to 5,000 kPa, preferably from about 100 kPa to 2,000 kPa and more preferably from about 300 kPa to about 1,000 kPa.

The process conditions may further include a weight hourly space velocity (WHSV) of from 0.1 to 25 hr⁻¹, preferably from about 0.5 to 15 hr⁻¹ and more preferably from about 2 to 12 hr⁻¹.

A molar ratio of the aromatic feed stream to oxygenate can be about 0.5:1 to 10:1, preferably from about 1:1 to 6:1 and more preferably from about 1.5:1 to 4:1. In an embodiment, the conditions may comprise a maximum temperature of less than about 650° C., a pressure of about 100 kPa to 1,000 kPa, and a toluene to methanol molar ratio of from about 1:2 to 6:1.

Any suitable oxygenates can be used. Suitable oxygenates include, for example, methanol, dimethylether, dimethyl carbonate, and mixtures thereof.

One or more suitable diluents can be used in the process. The one or more diluents can be added to the aromatic feed stream prior to addition to the methylation zone (i.e. reactor containing the methylation zone), or added directly to the methylation zone. Suitable diluents include H₂, H₂O, and combinations thereof. The molar ratio of diluent to aromatic feed stream and oxygenate feed stream (i.e. H₂O/(toluene+methanol)) can range from 0.1 to 3.0, preferably from 0.1 to 2.0 and most preferably from 0.2 to 1.5. In an aspect, the molar ratio can range from 0.1 to 3.0.

In one or more specific embodiments, the process for producing para-xylene includes reacting a toluene stream and a methanol stream in a toluene methylation zone operating under toluene methylation conditions comprising a maximum temperature of about 400° C. to about 675° C. and a pressure of about 10 kPa to 5,000 kPa in the presence of the MFI zeolites provided herein to produce a product stream comprising para-xylene.

In one or more other specific embodiments, the process for producing para-xylene includes reacting a toluene stream and a methanol stream in a toluene methylation zone operating under toluene methylation conditions comprising a maximum temperature of about 400° C. to about 675° C., a pressure of about 10 kPa to 5,000 kPa, a weight hourly space velocity of from 0.5 to 20 hr⁻¹ and a toluene to methanol molar ratio of from about 1:1 to 6:1, in the presence of a MFI zeolite provided herein to produce to produce a product stream comprising para-xylene.

### EXAMPLES

Embodiments discussed and described herein can be further described with the following examples. Although the following examples are directed to specific embodiments, they are not to be viewed as limiting in any specific respect.

In the examples that follow, ten (10) MFI zeolites, including Al-MFI-TPA, Fe-MFI-TPA, Ga-MFI-TPA, B-MFI-TPA, (B)—Al-MFI-EDA/TPA, (B)—Fe-MFI-EDA/TPA, (B)—Ga-MFI-EDA/TPA, B-MFI-EDA/TPA, Fe-MFI-EDA/TPA and Ga-MFI-EDA/TPA were synthesized.

While remaining selective towards p-X, these catalysts demonstrated total toluene methylation rates that varied as a function of the heteroatom used during synthesis. The MFI-TPA samples are denoted as T-MFI-TPA to denote tetra-n-propylammonium (TPA⁺ or simply, TPA) as the sole organic SDA in the synthesis solution. The MFI-EDA/TPA samples are denoted as T-MFI-EDA/TPA to denote ethylenediamine (EDA) and TPA were used as co-SDAs. The letter “T” represents the heteroatom precursor used (i.e., Al, Fe, Ga and/or B).

### Example 1: Synthesis and Characterization of T-MFI-TPA

The Al-MFI-TPA zeolites were synthesized by adding tetra-n-propylammonium hydroxide (TPAOH, 40 wt %, Alfa Aesar, 10 g 8.3 g), and deionized H₂O (18.2 MΩ, 33 g) in a perfluoroalkoxy alkane (PFA) container (Savillex Corp.) and stirring the solution under ambient conditions for 0.25 h. Next, aluminum hydroxide (Al(OH)₃, 99 wt %, 0.074 g) and NaOH (1 M solution in H₂O, 8.8 g) were added to the solution, and the mixture was stirred under ambient conditions for 0.25 h to homogenize the contents. Then, fumed silica (SiO₂, 99 wt %, 5.0 g) was added to the solution, and the mixture was stirred under ambient conditions for 24 h. The synthesis solution was then transferred to a 120 mL Teflon-lined stainless-steel autoclave and placed in a forced convection oven at 443 K and rotated at 40 rpm for 144 h.

The Fe-MF1-TPA zeolites were synthesized by the same protocols using Fe(NO₃)₃ (nonahydrate, Sigma-Aldrich, >99.9%, 0.49 g), TPAOH (40 wt %, Alfa Aesar, 10 g), NaOH (1 M solution in H₂O, 2.1 g), and deionized H₂O (18.2 MΩ, 17 g).

The Ga-MFI-TPA zeolites were synthesized by the same protocols as Al-MFI-TPA using Ga(NO₃)₃ (hydrate, Sigma-Aldrich, 99.9%, 0.16 g) instead of Al(OH)₃.

The B-MFI-TPA zeolites were synthesized using Boric acid (H₃BO₃, Sigma-Aldrich, >99.9%, 0.26 g), TPAOH (40 wt %, Alfa Aesar, 10 g), NaOH (1 M solution in H₂O, 10.1 g), deionized H₂O (18.2 MΩ, 24 g), and fumed silica (SiO₂, Cabosil, Sigma-Aldrich, 99 wt %, 6.0 g).

### Example 2: Synthesis and Characterization of B-T-MFI-EDA/TPA

These MFI zeolites were synthesized by adding EDA (99.5 wt %, Sigma-Aldrich) and H₃BO₃ (99.5 wt %, Sigma-Aldrich) to deionized H₂O (18.2 MΩ) in a PFA container and stirring the solution under ambient conditions for 0.25 h. Next, the heteroatom precursor (i.e., aluminum hydroxide (Al(OH)₃, 99 wt %), Fe(NO₃)₃ (nonahydrate, >99.9%), or Ga(NO₃)₃ (hydrate, 99.9%)) and TPAOH (40 wt %, Alfa Aesar) were added to the EDA-containing solution, and the mixture was stirred under ambient conditions for 0.25 h to homogenize the contents. The addition of Al, Ga, or Fe heteroatom precursors was not performed to prepare the boron-only sample (B-MFI-EDA/TPA); but instead, the boric acid elemental ratio was increased from 0.04 to 0.4 in the synthesis mixture. Finally, colloidal silica (Ludox HS-40, 40 wt %, Sigma-Aldrich) was added to the mixture and stirred for 24 h under ambient conditions. The synthesis solution was then transferred to a 120 mL Teflon-lined stainless-steel autoclave and placed in a forced convection oven at 448 K and rotated at 50 rpm for 120 h.

For the (B)—Al-MFI-EDA/TPA, (B)—Fe-MFI-EDA/TPA, and (B)—Ga-MFI-EDA/TPA samples, synthesis gels with a molar ratio of 0.3 EDA/0.04 H₃BO₃/0.02 Y/1 SiO₂/0.02 TPAOH/10.2 H₂O were prepared, where Y is the heteroatom precursor which was aluminum hydroxide (Al(OH)₃, 99 wt %), Fe(NO₃)₃ (nonahydrate, >99.9%), or Ga(NO₃)₃ (hydrate, 99.9%), respectively.

### Example 3: Synthesis and Characterization of T-MFI-EDA/TPA

These MFI zeolites were synthesized by adding EDA (99.5 wt %, Sigma-Aldrich) to deionized H₂O (18.2 MΩ) in a PFA container and stirring the solution under ambient conditions for 0.25 h. Next, the heteroatom precursor (i.e., Fe(NO₃)₃ (nonahydrate, >99.9%), or Ga(NO₃)₃ (hydrate, 99.9%)) and TPAOH (40 wt %, Alfa Aesar) were added to the EDA-containing solution, and the mixture was stirred under ambient conditions for 0.25 h to homogenize the contents. Finally, colloidal silica (Ludox HS-40, 40 wt %, Sigma-Aldrich) was added to the mixture and stirred for 60 h at 323 K. The synthesis solution was then transferred to a 120 mL Teflon-lined stainless-steel autoclave and placed in a forced convection oven at 448 K and rotated at 50 rpm for 120 h.

For the Fe-MFI-EDA/TPA and Ga-MFI-EDA/TPA samples, synthesis gels with a molar ratio of 0.3 EDA/0.01 Y/1 SiO₂/0.02 TPAOH/10.2 H₂O were prepared, where Y is the heteroatom precursor, which was Fe(NO₃)₃ (nonahydrate, >99.9%), or Ga(NO₃)₃ (hydrate, 99.9%), respectively.

The zeolite samples of Examples 1, 2 and 3 were recovered from autoclaves and washed with deionized water (18.2 MΩ) and acetone (Sigma-Aldrich, 99.9 wt %) in alternating steps until the pH of the supernatant remained constant between washes. Solids then were recovered via centrifugation, dried at 373 K under stagnant air for 24 h, and then treated in flowing dry air (1.67 cm³ s⁻¹ g_{cat}⁻¹, 99.999% UHP, Indiana Oxygen) at 853 K (0.0167 K s⁻¹) for 10 h. MFI zeolites were converted to NH₄-form via aqueous phase ion-exchange using 150 cm³ of a 1.0 M NH₄NO₃ solution (8.0 wt % in deionized water, 99.9 wt %, Sigma-Aldrich), followed by washing four times with deionized water (70 cm³ per g solids). NH₄-form zeolites were converted to their proton form via treatment in flowing dry air (1.67 cm³ s⁻¹ g_{cat}⁻¹, 99.999% UHP, Indiana Oxygen) at 773 K (0.0167 K s⁻¹) for 4 h.

The crystalline structure of the synthesized materials was determined from powder X-ray diffraction (XRD) patterns measured on a Rigaku SmartLab or an AntonParr XRDynamic 500 X-ray diffractometer with a Cu Kα source (λ=0.154 nm) operated at 1.76-2.0 kW. Diffraction patterns were measured from 4-40° 2θ. All patterns collected were consistent with the MFI topology. Zeolite micropore volumes were calculated from N₂ adsorption isotherms collected at 77 K for H-MFI samples in a Micromeritics 3-Flex Surface Area and Porosity Analyzer by finding the minimum of the semilogarithmic plot of ∂(V_{ads})/∂(ln(P/P₀)) versus ln(P/P₀). All micropore volumes were typical of highly crystalline MFI structure (ca. 0.14 cm³ g⁻¹).

Elemental compositions of the samples were analyzed using inductively coupled plasma-optical emission spectroscopy (ICP-OES) with a Thermo Scientific iCAP 7000 Plus Series ICP-OES. Aqueous samples were prepared by dissolving ca. 0.02 g of solid in 2.5 g of hydrofluoric acid (48 wt %, Alfa Aesar). After >24 h, 1 g of HNO₃ (70 wt %, Sigma-Aldrich) was added and diluted with 50 g of deionized water.

Ammonia temperature-programmed desorption (TPD) experiments were performed using a Micromeritics AutoChem II 2920 Chemisorption analyzer connected to a residual gas analyzer (MKS Cirrus). Methods to titrate protons at T heteroatoms (H⁺_{T}) in (i.e., Al, Fe, or Ga) in (B)-T-MFI-EDA/TPA zeolites were adapted from Kester et al.⁶ In short, NH₄-form (B)-T-MFI-EDA/TPA samples (0.02-0.05 g, sieved to 180-250 μm) were supported between two quartz wool plugs in a U-shaped quartz cell and placed in a clam-shell furnace. Samples were then heated under flowing He (15 cm³ s⁻¹ g_{cat}⁻¹, UHP, 99.999%, Indiana Oxygen) to 433 K (0.167 K s⁻¹) and held isothermally for 4 h to remove NH₃ bound to B heteroatoms. The temperature was then increased to 873 K (0.167 K s⁻¹) and evolved gases were sent to the RGA for quantification. H⁺ sites in the B-free T-MFI-TPA samples were quantified with similar methods without the 4 h 433 K isothermal hold step.

Aromatic methylation studies were conducted in a tubular packed-bed gas-phase quartz reactor (Quartz Scientific, Inc., 7 mm ID) passed through a three-zone furnace (Applied Test Systems) controlled by Watlow temperature controllers (EZ-ZONE). Bed temperatures were measured by a K-type thermocouple externally contacting the quartz tube. For a typical experiment, 60-180 mg of NH₄⁺-form MFI was pelleted and sieved to between 180 and 250 μm in diameter and diluted with 820-940 mg of quartz sand (Sigma-Aldrich, 180-250 μm), maintaining a total bed weight of 1 g. The bed was supported by two plugs of quartz wool (Thermo Scientific, fine, 4 μm), with the bottom of the bed eight inches from the reactor outlet. Pre-treatment to recover the H form and desorb water was first run by flowing 1.67 cm³ s⁻¹ of 5% O₂/Ar and ramping the temperature at 5 K min⁻¹ to 773 K, followed by a four-hour hold and a ramp down at 5 K min⁻¹ to the reaction temperature of 403 K, with a one-hour flush with 0.83 cm³ s⁻¹ of He before reactants were introduced.

Gas flow rates were metered with mass flow controllers (Parker 601) to give a total volumetric flow rate of 0.83 cm³ s⁻¹. DME (Matheson, CP, >99.5%) flow was adjusted to maintain 66 kPa, and an internal standard of 25% CH₄/Ar was introduced at 0.054 cm³ s⁻¹. Liquid aromatics were introduced by a syringe pump (KD Scientific Legato 100) connected to a heated tee (VICI, 473 K), vaporizing the liquid into the gas stream. The makeup gas to maintain the total flow rate was He (UHP, Indiana Oxygen). Reactor lines were maintained above 400 K with heat tracing to avoid reactant or product condensation. Product concentrations were quantified by an Agilent 7890B GC with a DB-WAX column (30 m, 0.32 mm, 0.50 μm, 7-inch cage). Flows were stabilized in a bypass stream and confirmed by GC before switching the flow to process through the reactor at time zero.

Measured elemental compositions and active (H⁺) site counts for the synthesized MFI zeolites are given in Table 1.

TABLE 1
Catalyst properties of synthesized MFI

| | | | Active site density, |
|---|---|---|---|
| Sample | Si/B ^{a} | Si/T ^{b} | mol H⁺_{T}/g_{cat} |
| Al-MFI-TPA | ∞ (B-free) | 43 | 2.81 × 10⁻⁴ |
| Fe-MFI-TPA | ∞ (B-free) | 83.6 | 1.96 × 10⁻⁴ |
| Ga-MFI-TPA | ∞ (B-free) | 117 | 1.38 × 10⁻⁴ |
| B-MFI-TPA | 67 | ∞ (T-free) | 1.29 × 10⁻⁴ |
| (B)-Al-MFI-EDA/TPA | 87 | 63.9 | 2.24 × 10⁻⁴ |
| (B)-Fe-MFI-EDA/TPA | 62.1 | 62.9 | 2.12 × 10⁻⁴ |
| (B)-Ga-MFI-EDA/TPA | 161 | 99 | 2.86 × 10⁻⁴ |
| B-MFI-EDA/TPA | 33 | ∞ (T-free) | 5.94 × 10⁻⁴ |

^{a} Determined from ICP-OES elemental analysis
^{b} Determined from ICP-OES elemental analysis, where T is Al, Fe, or Ga
^{c} Determined from liquid-phase NH₄⁺ ion exchange followed by NH₃ TPD. The reported H⁺ sites are deconvoluted from B heteroatom contributions and represent only the H⁺ sites associated with T atoms.⁶

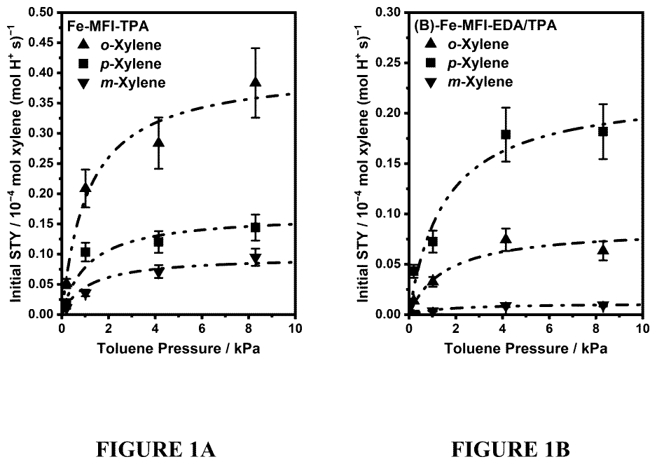

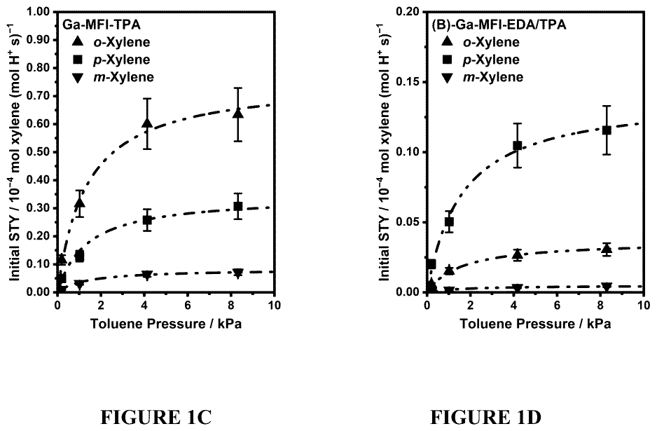

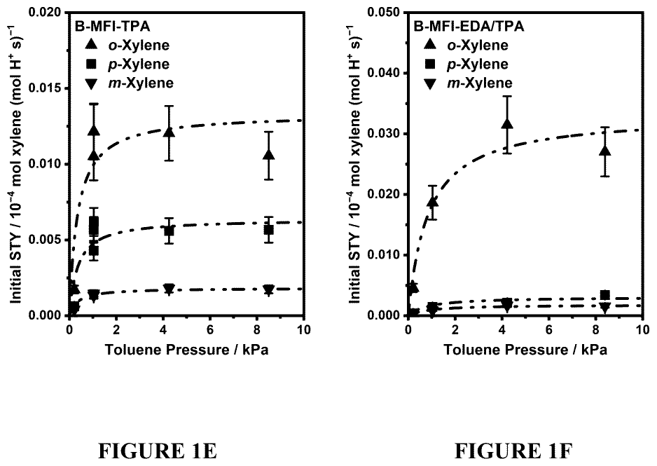

Initial product site-time yields (STY) are summarized in FIGS. 1A-1F. FIGS. 1A-1F depict individual xylene formation rates as a function of aromatic pressures on Fe-MFI-TPA (FIG. 1A), (B)—Fe-MFI-EDA/TPA (FIG. 1B), Ga-MFI-TPA (FIG. 1C), (B)—Ga-MFI-EDA/TPA (FIG. 1D), B-MFI-TPA (FIG. 1E), and B-MFI-EDA/TPA (FIG. 1F). Trendlines were fitted to Equation 1:

$$\frac{{r}_{i}}{[ {H}^{+} ]} = \frac{{k}_{i} {K}_{C} {P}_{i}}{1 + {K}_{C} {P}_{i}} \qquad Equation ( 1 )$$

Reaction conditions were: 130° C., DME partial pressure 66 kPa, space velocities of 0.0016-0.22 mol_{Toluene} mol_{H+}⁻¹s⁻¹.

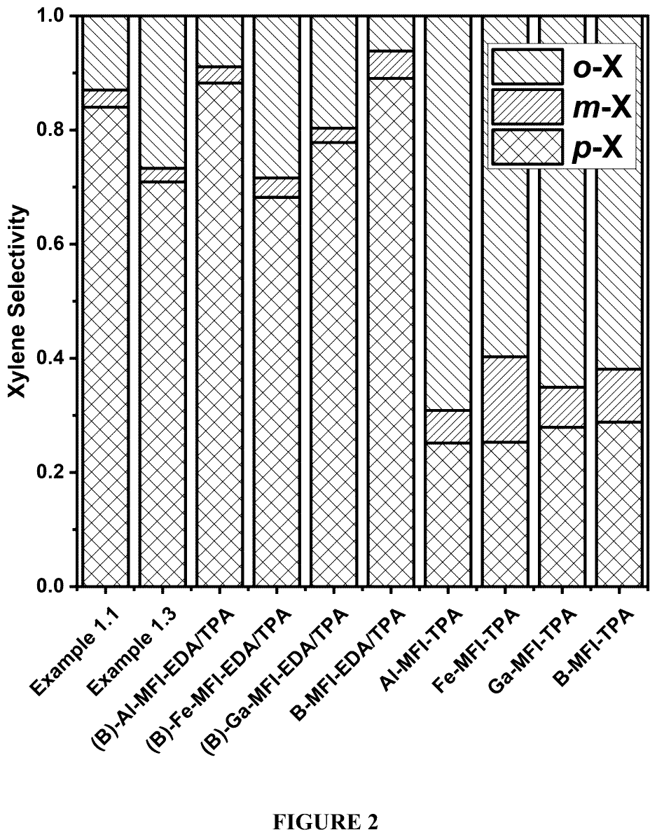

Rates within the range of 0-10 kPa toluene pressures demonstrated a transition from a first-order dependence on aromatic pressure to a zero-order dependence, which is captured by fitting Equation 1 to the experimental data. The TPA only samples containing Al, Fe, Ga, or B had higher rates for, and thus, selectivity towards, o-X. Conversely, the EDA/TPA samples showed suppressed rates for o-X and m-X formation. In addition, p-X formation rates in EDA/TPA samples did not show the same extent of suppression, and thus becoming the major product for (B)—Fe-MFI-EDA/TPA, (B)—Ga-MFI-EDA/TPA, and B-MFI-EDA/TPA. This trend was further captured in the xylenes selectivity in the zero-order region across the Al, Fe, Ga, and B samples shown in FIG. 2.

FIG. 2 depicts xylenes zero-order selectivity across the different synthesis methods (EDA/TPA) and heteroatoms (Al, Fe, Ga, B). Reaction conditions were: 130° C., DME partial pressure 66 kPa, space velocities of 0.0016-0.22 mol_{Toluene} mol_{H+}⁻¹s⁻¹. According to the results depicted in FIG. 2, selectivity was not a function of the heteroatom but instead was exclusively dependent on the SDA. The EDA/TPA family of samples provided p-X selectivity of >65%, while the TPA family provided p-X selectivity of <35%. m-X was a minor product across all samples. In comparison, the B-free Al-MFI-EDA/TPA and (B)—Al-MFI-EDA/TPA samples demonstrated selectivity towards p-X in the aluminosilicate case was not dependent on the supporting heteroatoms, but is correlated with the interactions between the primary trivalent element (Al) and the SDA.

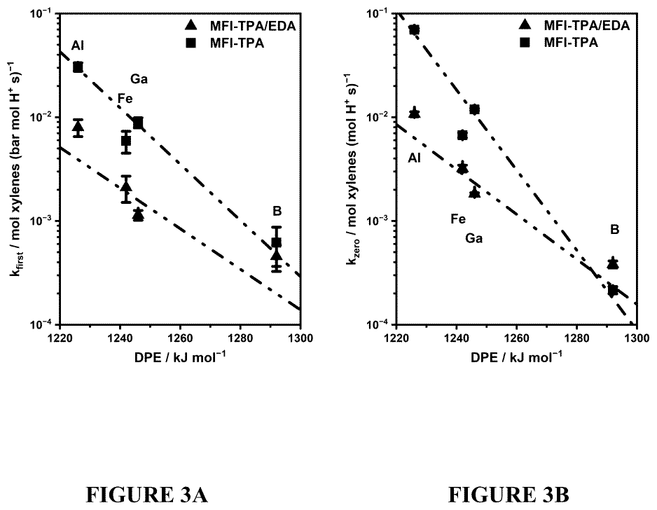

Total xylenes formation rates in both kinetic regimes, by heteroatom, are plotted in FIGS. 3A and 3B. FIG. 3A depicts first-order rate constants for total xylenes formation versus the DPE of each framework heteroatom. Reaction conditions were: 130° C., DME partial pressure 66 kPa, space velocities of 0.0016-0.22 mol_{Toluene} mol_{H+}⁻¹ s⁻¹. FIG. 3B depicts zero-order rate constants for total xylenes formation versus the DPE of each framework heteroatom. Reaction conditions were: 130° C., DME partial pressure 66 kPa, space velocities of 0.0016-0.22 mol_{Toluene} mol_{H+}⁻¹ s⁻¹.

The deprotonation energies (DPE) for framework heteroatoms were taken from US Publication No. 2023/0278017A1. The dependence on acid strength was similar between the EDA/TPA and TPA only family of samples, with EDA/TPA rates lower by a factor of 2.5-4.3 for all three pairs of heteroatom catalysts. Zero-order rates on (B)—Fe-MFI-EDA/TPA were 2.4× lower than those of (B)—Al-MFI-EDA/TPA, while those on (B)—Ga-MFI-EDA/TPA were 2.6× lower.

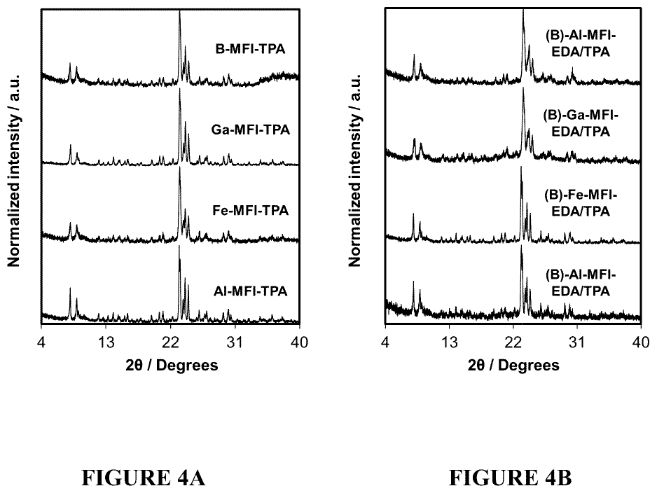

FIGS. 4A and 4B show the X-ray diffraction patterns for the T-MFI-TPA and (B)-T-MFI-EDA/TPA samples, respectively, that were synthesized with various framework heteroatoms. FIG. 4A shows X-ray diffraction patterns that confirm that the MFI framework topology was crystallized with TPA various framework heteroatoms. FIG. 4B shows X-ray diffraction patterns that confirm that the MFI framework topology was crystallized with TPA and EDA with various framework heteroatoms in the co-presence of B.

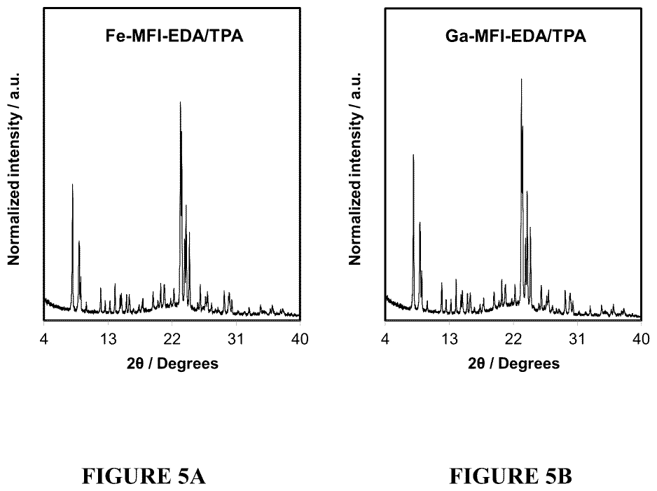

FIGS. 5A and 5B show the X-ray diffraction patterns for the Fe-MFI-EDA/TPA and Ga-MFI-EDA/TPA samples, respectively, synthesized without the addition of H₃BO₃ precusor. FIGS. 5A and 5B show X-ray diffraction patterns that confirm that the MFI framework topology was crystallized with TPA and EDA with various framework heteroatoms in the absence of B.

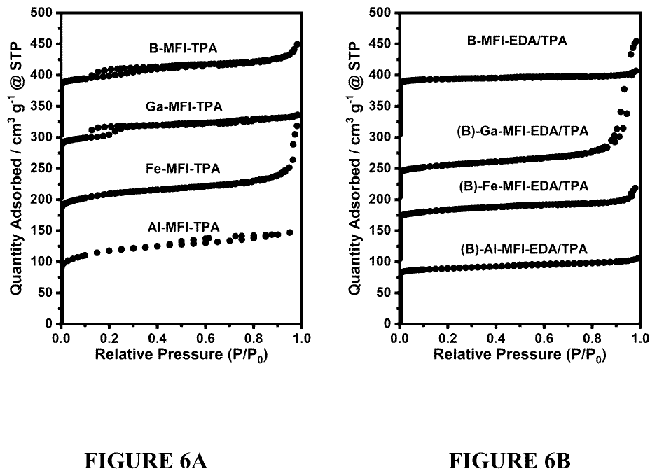

FIGS. 6A and 6B show the N₂ physisorption measurements at 77 K measured on T-MFI-TPA and (B)-T-MFI-EDA/TPA samples, respectively, synthesized with various framework heteroatoms. FIG. 6A shows N₂ physisorption isotherms that confirm that the MFI framework topology was crystallized with TPA various framework heteroatoms. FIG. 4B shows N₂ physisorption isotherms that confirm that the MFI framework topology was crystallized with TPA and EDA with various framework heteroatoms in the co-presence of B.

Additional aspects of the present invention include any of the following numbered embodiments below:
Embodiment 1: A process for selective aromatic methylation using MFI zeolites, comprising: contacting one or more aromatics and one or more oxygenates with a MFI zeolite comprising less than 30 wt % aluminum at reaction conditions sufficient to produce a product comprising one or more xylenes, wherein the MFI zeolite is obtained by: combining one or more structure directing agents (SDA) and water to form an aqueous solution; adding one or more heteroatoms, T, selected from the group consisting of boron, gallium, iron, and mixtures thereof to the aqueous solution to form an intermediate agent; adding a source of silicon to the intermediate agent to form a synthesis gel; and crystallizing the synthesis gel to form the MFI zeolite.

Embodiment 2: The process according to Embodiment 1, wherein the product comprises para-xylene.

Embodiment 3: The process according to Embodiments 1 or 2, wherein the MFI zeolite has a SiO₂/Al₂O₃ ratio of from about 80 to about 550.

Embodiment 4: The process according to any Embodiment 1 to 3, wherein the molar ratio of Si/T is between about 20 and about 100.

Embodiment 5: The process according to any Embodiment 1 to 4, wherein the one or more aromatics are benzene, toluene, or both.

Embodiment 6: The process according to any Embodiment 1 to 5, wherein the one or more oxygenates are selected from methanol, dimethyl ether, dimethyl carbonate and mixtures thereof.

Embodiment 7: The process according to any Embodiment 1 to 6, wherein the reaction conditions comprise a temperature of from about 400° C. to about 675° C.

Embodiment 8: The process according to any Embodiment 1 to 7, wherein the reaction conditions comprise a pressure of from about 10 kPa to 5,000 kPa.

Embodiment 9: The process according to any Embodiment 1 to 8, further comprising conducting an ion-exchange treatment to remove unreacted reagents, and then recovering an acid-form of the MFI zeolite.

Embodiment 10: The process according to any Embodiment 1 to 9, wherein the reaction conditions comprise an aromatic to oxygenate molar ratio of from about 0.5:1 to 10:1.

Embodiment 11: The process according to any Embodiment 1 to 10, wherein the reaction conditions comprise a weight hourly space velocity (WHSV) of from 0.1 to 20 hr⁻¹.

Embodiment 12: The process according to any Embodiment 1 to 11, wherein the one or more structure directing agents (SDAs) are selected from the group consisting of tetrapropylammonium (TPA), ethylenediamine (EDA), 1,4-Diazabicyclo[2.2.2]octane (DABCO), methylamine (MA), tert-butylamine, n-butylamine, tripropylamine, dipropylamine, tetraethylammonium, 1,2,4-Trimetyl-1H-pyrazole-2-ium (1,2,4-TMP), pentaertythritol (PET), and combinations thereof.

Embodiment 13: The process according to any Embodiment 1 to 12, wherein the one or more structure directing agents (SDAs) consists of tetrapropylammonium (TPA) and ethylenediamine (EDA).

Embodiment 14: The process according to any Embodiment 1 to 13, wherein the one or more structure directing agents (SDAs) consist essentially of tetrapropylammonium (TPA) and ethylenediamine (EDA).

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, meaning the values take into account experimental error, machine tolerances and other variations that would be expected by a person having ordinary skill in the art.

The foregoing has also outlined features of several embodiments so that those skilled in the art can better understand the present disclosure. Those skilled in the art should appreciate that they can readily use the present disclosure as a basis for designing or modifying other methods or devices for carrying out the same purposes and/or achieving the same advantages of the embodiments disclosed herein. Those skilled in the art should also realize that such equivalent constructions do not depart from the spirit and scope of the present disclosure, and that they can make various changes, substitutions, and alterations herein without departing from the spirit and scope of the present disclosure, and the scope thereof is determined by the claims that follow.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A process for selective aromatic methylation using MFI zeolites, comprising:
contacting one or more aromatics and one or more oxygenates with a MFI zeolite comprising less than 30 wt % aluminum at reaction conditions sufficient to produce a product comprising one or more xylenes,
wherein the MFI zeolite is obtained by:
combining one or more structure directing agents (SDA) and water to form an aqueous solution;
adding one or more heteroatoms, T, selected from the group consisting of boron, gallium, iron, and combinations thereof to the aqueous solution to form an intermediate agent;
adding a source of silicon to the intermediate agent to form a synthesis gel; and
crystallizing the synthesis gel to form the MFI zeolite.

2. The process of claim 1, wherein the product comprises para-xylene.

3. The process of claim 1, wherein the MFI zeolite has a SiO₂/Al₂O₃ ratio of from about 80 to about 550.

4. The process of claim 1, wherein the molar ratio of Si/T is between about 20 and about 100.

5. The process of claim 1, wherein the one or more aromatics are benzene, toluene, or both.

6. The process of claim 1, wherein the one or more oxygenates are selected from methanol, dimethyl ether, dimethyl carbonate and mixtures thereof.

7. The process of claim 1, wherein the reaction conditions comprise a temperature of from about 400° C. to about 675° C.

8. The process of claim 1, wherein the reaction conditions comprise a pressure of from about 10 kPa to 5,000 kPa.

9. The process of claim 1, further comprising conducting an ion-exchange treatment to remove unreacted reagents, and then recovering an acid-form of the MFI zeolite.

10. The process of claim 1, wherein the reaction conditions comprise an aromatic to oxygenate molar ratio of from about 0.5:1 to 10:1.

11. The process of claim 1, wherein the reaction conditions comprise a weight hourly space velocity (WHSV) of from 0.1 to 20 hr⁻¹.

12. The process of claim 1, wherein the one or more structure directing agents (SDAs) are selected from the group consisting of tetrapropylammonium (TPA), ethylenediamine (EDA), 1,4-Diazabicyclo[2.2.2]octane (DABCO), methylamine (MA), tert-butylamine, n-butylamine, tripropylamine, dipropylamine, tetraethylammonium, 1,2,4-Trimetyl-1H-pyrazole-2-ium (1,2,4-TMP), pentaertythritol (PET), and combinations thereof.

13. The process of claim 1, wherein the one or more structure directing agents (SDAs) consists of tetrapropylammonium (TPA) and ethylenediamine (EDA).

14. The process of claim 1, wherein the one or more structure directing agents (SDAs) consist essentially of tetrapropylammonium (TPA) and ethylenediamine (EDA).
